(19) United States
(12) Patent Application Publication
(10) Pub. No.: US 2023/0279453 A1
(43) Pub. Date: Sep. 7, 2023

(54) Improved Cellulose to Cellobiose Conversion Process

(71) Applicant: SixRing Inc., Calgary (CA)

(72) Inventors: Alejandra ENRIQUEZ, Calgary (CA); Julie GREER, Calgary (CA); Alexandra OSTASZEWSKI, Calgary (CA); Markus WEISSENBERGER, Calgary (CA)

(21) Appl. No.: 18/117,163

(22) Filed: Mar. 3, 2023

(30) Foreign Application Priority Data
Mar. 4, 2022 (CA) 3151040

(51) Int. Cl. C12P 19/12; C12P 19/02; C12P 7/14; C12N 1/20; C12P 19/14

(52) U.S. Cl. CPC C12P 19/12; C12P 19/02; C12P 7/14; C12N 1/20; C12P 19/14

(57) ABSTRACT

A process to hydrolyze cellulose into cellobiose comprising the following steps:
providing a reaction vessel;
providing a Cellulomonas uda (ATCC 491) inoculum;
exposing said Cellulomonas uda (ATCC 491) bacterium to a source of cellulose having a kappa number of less than 10 in an aqueous medium of pH of about 8 at a temperature ranging from 30° C. to 35° C. for a period of time ranging from 14 to 42 days;
exposing the cellobiose to a bacterium or fungi or yeast, or combination which converts cellobiose to glucose or ethanol.

### FIELD OF THE INVENTION

The present invention is directed to a novel process to generate cellobiose from cellulose, more specifically, a process which uses a specific cellulose in order to generate substantial improvement in conversion to cellobiose.

### BACKGROUND OF THE INVENTION

Biofuel is increasingly becoming a necessity in order to wean off the human consumption of fossil fuels in aspects of everyday life, transport and home heating being the largest two industries of focus. As an alternative energy source to oil and coal, the main feedstock for biofuel production is starch which can yield its sugar much more readily than cellulose. This is due to the difference in structure as starch links glucose molecules together through alpha-1,4 linkages and cellulose links glucose with beta-1,4 linkages. The beta-1,4 linkages allow for crystallization of the cellulose, leading to a more rigid structure which is more difficult to break down.

The limitation that comes from solely concentrating the biofuel on extracting the sugars from starches prevents the utilization of the larger portion of biomass which comes in the form of lignocellulosic biomass (contains lignin, cellulose and hemicellulose) present in almost every plant on earth. A delignification reaction allows the recovery of cellulose from those lignocellulosic plants. Once the cellulose is separated from the other two biomass constituents i.e., lignin, and hemicellulose, further degradation of the cellulose generates cellobiose and/or glucose which can be further processed to bio-ethanol.

Seen as a sustainable alternative to gasoline and with the goal of alleviating many countries' dependence on foreign oil, the biofuel industry is still hampered by its dependence on corn or sugar cane as their main sources of fuel, as they are both rich in starch. It is estimated that about a third of all corn production in the U.S. is directed to the ethanol fuel production. This is a situation which has disastrous consequences when the prices of gasoline go so low as to make corn-based biofuel unsustainable on a price view point.

Many corn-ethanol producers have recently temporarily closed plants in the U.S. which has had as direct impact of overall lower corn purchases across the country and a glut of unused corn which requires storage.

Across the world, many other large ethanol-producing countries, including China and Brazil, have shown some struggles in ethanol production from biomass as many companies are carrying large debts from the implementation of such processes and large plants have been to shut down or decrease production.

In Asia, palm oil prices have recently increased to their highest levels in years, which, in turn, will hamper the ability of Indonesia and Malaysia to produce local biofuel. Oil palm trunk is a valuable and plentiful resource in those countries to generate biofuels and biochemicals. Oil palm trunk contains a large amount of starch which is more readily solubilized in water, compared to cellulose. Starch can then be heated and hydrolyzed to glucose by amylolytic enzymes without pre-treatment. However, the conventional Oil palm trunk treatment requires high capital and operational costs and is therefore prohibitive to market entry. Moreover, the treatment carries a high probability of microbial contamination during starch processing.

In Europe, where over 70% of the rapeseed oil produced is used to manufacture biofuel, there are concerns about the vagaries in demand for blending biofuels which has caused some groups to substantially decrease their ethanol production.

To pivot from starches to cellulose for the production of glucose is preferable as it will provide near-unlimited amount of feedstock from waste biomass and reduce the competition with food source feedstock to generate glucose. However, the costs to do so are currently prohibitive. Cellulosic ethanol as it is called relies on the non-food part of a plant to be used to generate ethanol. This would allow the replacement of the current more widespread approach of making bioethanol by using corn or sugarcane. The diversity and abundance of these types of cellulose-rich plants would allow to maintain food resources mostly intact and capitalize on the waste generated from these food resources (such as cornstalk) to generate ethanol. Other cellulose sources such as grasses, algae and even trees fall under the cellulose-rich biomass which can be used in generating ethanol if a commercially viable process is developed.

The reason why starches are preferred to cellulose-rich sources to generate ethanol is that extraction of glucose from cellulose is substantially more difficult and resource intensive. To better understand the difference which raises this difficulty it is worthwhile pointing the similarities and differences between starch and cellulose.

Cellulose and starch are polymers which have the same repeat units of glucose. However, the differences between starch and cellulose can be seen in the repeating glucose monomers that are connected to one another. In starch, the glucose monomers are oriented in the same direction. In cellulose, each successive glucose monomer is rotated 180 degrees in respect of the previous glucose monomer. This, in turn, ensures that the bonds between each monomeric glucose differs between starch and cellulose. In starch, the bonds (otherwise known as links) are referred to as alpha 1,4 linkages, in cellulose these bonds are referred to as beta 1,4 linkages.

The difference between these bonds impacts the characteristics of starch and cellulose. Starch can dissolve in warm water while cellulose does not. Starch can be digested by humans, cellulose cannot. Starch is weaker than cellulose partly due to the fact that its structure is less crystalline than cellulose. Starch is, at its core, a method for plants to store energy, therefore extracting sugars from starch is much easier than to do so from cellulose as the latter's core function is to provide structural support,

As the main component of lignocellulosic biomass, cellulose is a biopolymer consisting of many glucose units connected through β-1,4-glucosidic bonds (see FIG. 1). D-glucose is the building block of many polysaccharides, including cellulose. Glucose has two isomers: α-glucose (present in starches as branched polymers) and β-glucose (present in cellulose as repeating units of β-glucose subunits connected via a β-1,4-glucosidic bond with one β-glucose monomer rotated by 180 degrees relative to its neighbour). A cellulose molecule can comprise between hundreds to thousands of glucose units. Since the cellulose molecules are linear, due in part to intermolecular hydrogen bonding, neighboring cellulose molecules can be very closely packed and, in turn, provide the structural strength needed to support plants.

The hydrolysis of cellulose is achieved by cleaving the β-1,4-glucosidic bonds by exposing such to acid solutions. Hydrolysis of cellulose results in the generation of glucose and other oligosaccharides. Many different types of acids, such as HCl and H₂SO₄, have been used in the past to achieve this. The use of one of these acids usually results in at least one of the following drawbacks: corrosion of the reaction vessel; difficulty of disposing of the discharged reactants; and others.

### Hydrolysis of Cellulose

The hydrolysis of cellulose is the rate limiting step in the conversion of cellulose into biofuel. The processes currently using cellulose as a starting material for bioethanol production require the conversion of cellulose into cellobiose, then glucose, prior to the ultimate generation of ethanol. The fermentation of glucose using yeast is what leads to the production of ethanol. While that last step in biofuel production has been mastered for some time, the rate limiting step is the most crucial one and one which hinders a wider acceptance of biofuels. The difficulty in overcoming this conversion of cellulose into glucose lies with the fact that cellulose has a crystalline structure which renders its conversion to glucose quite difficult because of the close packing of multiple cellulose polymers. This close packing imparts on cellulose it's inherent stability under a variety of chemical conditions. Cellulose polymers are generally insoluble in water, as well as a number of organic solvents. Cellulose is also generally insoluble when exposed to weak acids or bases.

In general, there are two main approaches to hydrolyze cellulose: chemical and enzymatic. The chemical method resorts to the use of concentrated strong acids to hydrolyze cellulose under conditions of high temperature and pressure. The biofuel industry is generally reticent to use chemically hydrolyzed cellulose because of the presence of toxic by-products in the resulting glucose. These by-products, if introduced in the fermentation step, will negatively affect the delicate balance of the fermenting yeast.

### Cost of Enzymatic Hydrolysis

It is known that the costs to extract biofuel from cellulose are higher than when doing so from starch. It is estimated that, on average, depending on location and availability of biomass, the cost for cellulose conversion is about 50% more that starch conversion to glucose. This means that there currently is a clear barrier to producers for using cellulose rather than corn or other starch resources to generate glucose from biomass.

It is generally understood that roughly half of the total cost of producing biofuel from cellulose stems from the price of the enzymes (cellulases). The generation of enzymes for enzymatic hydrolysis of cellulose is a time-consuming process and large volumes of enzyme are required to render the process commercially viable. Approximately, 25 grams of enzyme is required to process 1 kg of cellulose. One possible approach is to improve the rate of the hydrolysis reaction which, in turn, would result in a decrease in the overall cost of the process.

The enzymatic approach to hydrolyzing cellulose uses enzymes to carry out the hydrolysis reaction. Enzymes, such as cellulases (comprising Endo-1,4-B-glucanases; Exo-1,4-B-glucanases; and B-glucosidases) require extensive controls in place to maximize the reaction rates the enzymatic approach is expected to provide. Temperature, pH, salinity, concentration of substrate and product are all factors that may affect enzyme activity. Small deviations from the enzyme's optimal conditions will result in loss of function. The conversion of cellulose to glucose is done by a few different enzymes: Endo-1,4-B-glucanases; Exo-1,4-B-glucanases; and B-glucosidases, all of which have specific environmental conditions which must be met. These controls render the process cost prohibitive in some cases and/or limiting in their implementation.

PCT patent application WO9640970 (A1) discloses a method of producing sugars from materials containing cellulose and hemicellulose comprising: mixing the materials with a solution of about 25-90% acid by weight thereby at least partially decrystallizing the materials and forming a gel that includes solid material and a liquid portion; diluting said gel to an acid concentration of from about 20% to about 30% by weight and heating said gel to a temperature between about 80° C. and 100° C. thereby partially hydrolyzing the cellulose and hemicellulose contained in said materials; separating said liquid portion from said solid material, thereby obtaining a first liquid containing sugars and acid; mixing the separated solid material with a solution of about 25-90% acid until the acid concentration of the gel is between about 20-30% acid by weight and heating the mixture to a temperature between about 80° C. and 100° C. thereby further hydrolyzing cellulose and hemicellulose remaining in said separated solid material and forming a second solid material and a second liquid portion; separating said second liquid portion from said second solid material thereby obtaining a second liquid containing sugars and acid; combining the first and second liquids; and separating the sugars from the acid in the combined first and second liquids to produce a third liquid containing a total of at least about 15% sugar by weight, which is not more than 3% acid by weight.

U.S. Pat. No. 4,496,656A describes a process for production of cellulase according to the present invention thus comprises culturing a cellulase-producing microorganism belonging to Cellulomonas uda CB4 in a cellulose-containing medium, and recovering the cellulase produced from the culture broth. According to the present invention, because the bacteria belonging to Cellulomonas uda CB4 is capable of producing a cellulase having a high activity, not found in the reports of the prior art, in a culture medium, it is possible to produce a cellulase having a high crystalline cellulose decomposing activity comparable to those produced from a mold within a short cultivation period of two days.

In the paper entitled ‘Glucose production from cellulose through biological simultaneous enzyme production and saccharification using recombinant bacteria expressing the β-glucosidase gene’ by Ichikawa S. et al, (J Biosci Bioeng. 2019 March;127(3):340-344), there is disclosed a cellulosic biomass saccharification technologies. Glucose was produced by the hydrolysis of 100 g/L Avicel cellulose for 10 days through biological simultaneous enzyme production and saccharification (BSES), and the product yield was similar to that obtained through BSES with purified β-glucosidase supplementation.

In the paper entitled ‘A novel facile two-step method for producing glucose from cellulose’ (Bioresource Technology Volume 137, June 2013, Pages 106-110) a two-step acid-catalyzed hydrolysis methodology is disclosed where cellulose is hydrolyzed to glucose with high yield and selectivity under mild conditions. Its approach involves a multi-step hydrolysis, comprising as first step, the depolymerization of microcrystalline cellulose in phosphoric acid to cellulose oligomer at 50° C. The second step involves the precipitation of the oligomer by ethanol and subsequent hydrolysis with dilute sulfuric acid.

In the paper entitled ‘Dilute-acid Hydrolysis of Cellulose to Glucose from Sugarcane Bagasse’ from Dussan et al. (CHEMICAL ENGINEERING TRANSACTIONS VOL. 38, 2014), there is disclosed a method of generating ethanol through the hydrolysis of cellulose. Sugarcane bagasse is used as a substrate for ethanol production, optimum conditions for acid hydrolysis of cellulose fraction were assessed. The glucose thus generated was fermented to ethanol using the yeast (Scheffersomyces stipitis).

The hydrolysis of cellulose is, as seen from the above, limited by the structure of cellulose itself but also by the approaches taken to degrade it into a biofuel. The production of a robust, low-cost process from cellulose has not yet been achieved.

U.S. Pat. No. 9,663,807 discloses the preparation of ethanol by using lignocellulosic biomass such as corn stover which is pre-treated to remove C5 compounds (derived from hemicellulose), to leave C6 solids to be subsequently subjected to a simultaneous saccharification and fermentation (SSF) process. It was noted that simultaneous saccharification and fermentation could be performed at temperatures suitable for ethanol production by the yeast (e.g., about 37° C.) but this, in turn, was less than optimal for the cellulase enzyme. Consequently, the yields from such enzymes were lower because their activity was impeded by the presence of lignin on which cellulase enzymes could bind. In that case, it was discovered that addition of a lignin-binding agent, such as clarified thin stillage and/or Anaerobic Membrane Bioreactor (AnMBR) effluent could result in increased glucose yield during enzyme hydrolysis.

In light of the above, there is a profound need to develop a process for biofuel generation from waste biomass as an abundant and untapped source of renewable biofuels that does not compete with a food source, such as corn. In that respect, a lignocellulosic biomass from which cellulose is extracted is much more highly attractive as it will leave food sources available to fulfill their primary intended purpose and yet still generate a substantial cellulosic yield. The aforementioned is also substantiated with the tremendous efforts to convert waste biomass to biofuels using different approaches which have almost all failed to achieve this goal for subsequent conversion to glucose and ultimately, ethanol.

The inventors have surprisingly and unexpectedly found that the characteristics of the cellulose obtained from a specific type of delignification approach have a substantial impact on the downstream hydrolysis of said cellulose.

### SUMMARY OF THE INVENTION

According to an aspect of the present invention, there is provided a process to convert cellulose to cellobiose, said process comprising the steps of:
providing a reaction vessel;
providing a source of cellulose into said reaction vessel; wherein said source of cellulose having a kappa number of less than 10;
providing a Cellulomonas uda (ATCC 491) inoculum into said reaction vessel;
exposing said Cellulomonas uda (ATCC 491) to said source of cellulose in an aqueous medium of pH of about 8 at a temperature ranging from 30° C. to 35° C. for a period of time ranging from 14 to 42 days; and
optionally, recovering the supernatant comprising cellobiose.

Preferably, said source of cellulose has a particle size ranging between 30-50 μm.

Preferably, the process further comprises a step of exposing the cell supernatant to a bacterium or fungi or yeast, or combination, to convert cellobiose to glucose or ethanol.

According to a preferred embodiment of the present invention, the method of delignification of biomass material which yields a low lignin cellulose (also referred to as low kappa number cellulose and also referred to as Modified Caro's Acid (MCA) cellulose) used in the cellulose to cellobiose (and ultimately, glucose) conversion experiments comprise:
the source of cellulose is a lignocellulosic biomass delignified by exposure to a modified Caro's acid composition selected from the group consisting of: composition A; composition B and Composition C;

wherein said composition A comprises:
sulfuric acid in an amount ranging from 20 to 70 wt % of the total weight of the composition;
a compound comprising an amine moiety and a sulfonic acid moiety selected from the group consisting of: taurine; taurine derivatives; and taurine-related compounds; and
a peroxide;

wherein said composition B comprises:
an alkylsulfonic acid; and
a peroxide; wherein the acid is present in an amount ranging from 40 to 80 wt % of the total weight of the composition and where the peroxide is present in an amount ranging from 10 to 40 wt % of the total weight of the composition;

wherein said composition C comprises:
sulfuric acid;
a compound comprising an amine moiety;
a compound comprising a sulfonic acid moiety; and
a peroxide;
   for a period of time sufficient to remove substantially all of the lignin present on said biomass material. To alleviate the text, the above described process can be referred to hereinafter as the modified Caro's acid delignification process, as well as the obtained cellulose can be referred to as modified Caro's acid delignified cellulose or “MCA cellulose” to indicate the method of delignification employed to obtain said cellulose.

It is widely accepted that a kappa number is a reliable indication of lignin content in a pulp or cellulosic material. The higher the kappa number, the higher the lignin content is. The Kappa number is a measure of the degree of fibrous pulp digestion and can be applied to determine lignin content. Its value can vary from 0 to over 100, where 0 indicates a practically lignin-free pulp (such as that found in bleached pulp) and where a kappa number of 60 is usually attained with a standard unbleached pulp. When the kappa number is 60, this is a rough indication that the lignin content is about 9%, when the kappa number is about 20, this would indicate a lignin content of approximately 2.8-3.0%. When the kappa number is about 27, the lignin content is approximately 4.0%.

According to a preferred embodiment of the present invention, the process generates cellobiose (or glucose) from cellulose, employs a cellulose with low kappa number and low hemicellulose content, were said low kappa number and low hemicellulose content has the following characteristics: particle size ranging from β-1000 microns, a content of hemicellulose of less than 15%, preferably less than 10%; preferably ranging from 4 to 7 wt. % and more preferably less than 5 wt. % of the total weight of the cellulose; and a kappa number of less than 10, more preferably less that 5, and even more preferably, less than 2.

Preferably, said sulfuric acid, said compound comprising an amine moiety and a sulfonic acid moiety and said peroxide are present in a molar ratio of no more than 15:1:1. Also preferably, said sulfuric acid and said compound comprising an amine moiety and a sulfonic acid moiety are present in a molar ratio of no less than 3:1.

According to a preferred embodiment of the approach to obtain low lignin cellulose, said delignification lasts from 2 to 20 hours.

According to a preferred embodiment of the approach to obtain low lignin cellulose, said delignification is carried out at temperatures below 50° C. Preferably, the delignification is carried out at temperatures below 40° C.

According to a preferred embodiment of the approach to obtain low lignin cellulose, the resulting cellulose from the delignification process is dried, milled and sieved prior to hydrolysis.

According to another aspect of the present invention, there is provided a process to convert cellulose to glucose or ethanol, said process comprising the steps of:
providing a reaction vessel;
providing a source of cellulose into said reaction vessel; wherein said source of cellulose having a kappa number of less than 10;
providing a Cellulomonas uda (ATCC 491) inoculum into said reaction vessel;
exposing said Cellulomonas uda (ATCC 491) to said source of cellulose in an aqueous medium of pH of about 8 at a temperature ranging from 30° C. to 35° C. for a period of time ranging from 14 to 42 days, allowing sufficient exposure time to convert at least 90% of the cellulose into cellobiose and/or glucose;
optionally, recovering the supernatant comprising cellobiose and glucose; and
optionally also, further converting the cellobiose into glucose using a bacterium, fungi or combination of both.

Preferably, said source of cellulose has a particle size ranging between 30-50 μm.

Preferably, the temperature inside the reaction vessel during said exposure time does not exceed 60° C.

According to another aspect of the present invention, there is provided a process wherein said cellulose is characterized by an absence of prior exposure to bleaching chemicals selected from the group consisting of: sodium hydrosulphite (Na₂S₂O₄); hydrogen peroxide; pentasodium salt diethylenetriaminepentaacetic acid; amine borane (CH₃)₃CNH₂-BH₃; borane ammonia complex BH₃—NH3; sodium percarbonate; formamidine sulphinic acid; sodium perborate; and chlorine dioxide. The presence of such compounds on a bleached cellulose may have a negative impact on the Cellulomonas uda (ATCC491) bacterium and thus affect the yield of cellobiose. According to a preferred embodiment of the present invention, the process utilizes a low lignin content cellulose which allow the Cellulomonas uda (ATCC491) bacterium to efficiently metabolize the cellulose into cellobiose. The person skilled in the art will understand that, in the context of the present application, where there is a reference to bleaching of pulp, it is to be understood that the bleaching refers to a separate and distinct step of pulp processing. Consequently, the pulp used according to a preferred process of the present invention, is intended on being a pulp which has not undergone a separate bleaching step post-delignification. Such a treatment step is understood to not be economically viable when the ultimate goal of the cellulose is to be used to generate ethanol.

According to a preferred embodiment of the present invention, said cellulose has an aspect ratio of about 7.5. The aspect ratio of a particle, in this case, cellulose fibers, is determined by the ratio of its length over its width. In the above case, the particles are about 30 to 50 μm in length and about 4 μm in width.

According to a preferred embodiment of the present invention, said cellulose is substantially free of the hemicellulose which was present in the initial biomass material which underwent delignification to obtain a low lignin cellulose.

According to another aspect of the present invention, there is provided a process to obtain a bacterium having a high cellulase activity for a low kappa number cellulose, wherein said a low kappa number cellulose has the following characteristics: particle size ranging from 30-50 microns and a kappa number of less than 10, more preferably less than 5 and even more preferably, less than 2, wherein said process comprising the steps of:
providing a reaction vessel;
providing a Cellulomonas uda (ATCC 491) inoculum which has a cellulase enzyme activity when exposed to said low kappa number cellulose of less than 5 units/ml after said bacterium has been exposed to said low kappa number cellulose for 21 days;
exposing said Cellulomonas uda (ATCC 491) bacterium to said low kappa number cellulose in an aqueous medium of pH of about 8 at a temperature ranging from 30° C. to 35° C. for a period of time ranging from 14 to 42 days;
recovering the bacterium and repeating the step of exposing said bacterium to said low kappa number cellulose for a number of cycles of exposure and recovery until the cellulase activity of the enzyme reaches at least 8 units/ml when said bacterium is exposed to said low kappa number cellulose for at least 18 days, wherein said bacterium has been optimized for cellulose hydrolysis; and
optionally, recovering the optimized bacterium; and
optionally, removing the products and refeeding the culture for a continuous batch culture.

### BRIEF DESCRIPTION OF THE FIGURES

Features and advantages of embodiments of the present application will become apparent from the following detailed description and the appended figures, in which:

FIG. 1 is a depiction of glucose monomers present in a starch polymer and their α-1,4-linkages compared to glucose monomers present in a cellulose polymer and their β-1,4-linkages;

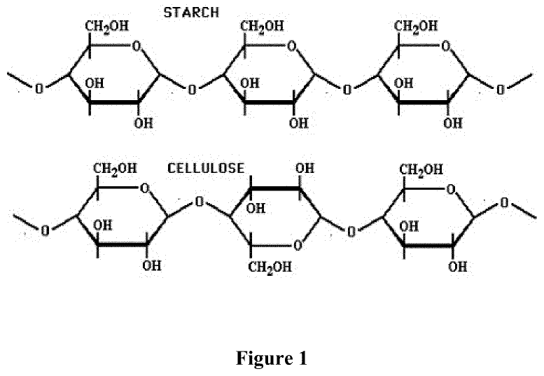

FIG. 2 illustrates a comparative graphical representation of the enzymatic activity of an adapted strain of Cellulomonas uda (ATCC 491), after a few generations of exposure to the Modified Caro's Acid (MCA) cellulose, upon exposure to fine grade and coarse grade delignified cellulose.

FIG. 3 illustrates a graphical representation of the enzymatic activity of the original strain of Cellulomonas uda (ATCC 491) in a continuous feed model.

FIG. 4 illustrates a graphical representation of the beta-glucanase enzymatic activity for various substrates obtained either by the kraft process or by exposure to a modified Caro's acid;

FIG. 5 illustrates a graphical representation of the beta-glucosidase enzymatic activity for various substrates obtained either by the kraft process or by exposure to a modified Caro's acid;

FIG. 6 illustrates a graphical representation of the ethanol yield for various substrates obtained either by the kraft process or by exposure to a modified Caro's acid;

### DETAILED DESCRIPTION OF THE INVENTION

The description that follows, and the embodiments described therein, is provided by way of illustration of an example, or examples, of particular embodiments of the principles of the present invention. These examples are provided for the purposes of explanation, and not limitation, of those principles and of the invention.

According to a preferred embodiment of the present invention, C. uda is more efficient at hydrolyzing the modified Caro's acid delignified cellulose (MCA cellulose) which has a kappa number of β-2 than a softwood cellulose with a kappa number of 27.

U.S. Pat. No. 4,496,656A describes a medium used to grow the bacteria used in the present invention. Several other media were tested, but this turned out to be the most suitable for the methods according to preferred embodiments of the present invention.

According to a preferred embodiment of the present invention, the process to hydrolyze cellulose into cellobiose comprises the following steps:
providing a reaction vessel;
providing an enzyme adapted to convert a source of cellulose having a particle size of 30-50 microns and a kappa number of less than 10, more preferably less than 5 and even more preferably, less than 2, in an aqueous medium of pH of about 8 at a temperature ranging from 30° C. to 35° C. for a period of time ranging from 14 to 42 days, into cellobiose;
exposing the cell supernatant to a bacterium or fungi or yeast, or combination, which converts cellobiose to glucose or ethanol.

According to a preferred embodiment of the present invention, the process to hydrolyze cellulose into cellobiose comprises the following steps:
providing a reaction vessel;
providing a Cellulomonas uda (ATCC 491) inoculum;
exposing said Cellulomonas uda (ATCC 491) to a source of cellulose having a particle size of 30-50 microns and a kappa number of less than 10, more preferably less than 5 and even more preferably, less than 2, in an aqueous medium of pH of about 8 at a temperature ranging from 30° C. to 35° C. for a period of time ranging from 14 to 42 days;
exposing the cell supernatant to a bacterium or fungi or yeast, or combination, which converts cellobiose to glucose or ethanol.

Cellulomonas uda (ATCC 491) is an aerobic bacterium isolated and characterized from compost. It is a gram-positive rod-shaped bacterium of the phylum Actinobacteria and is known for its ability to metabolize cellulose.

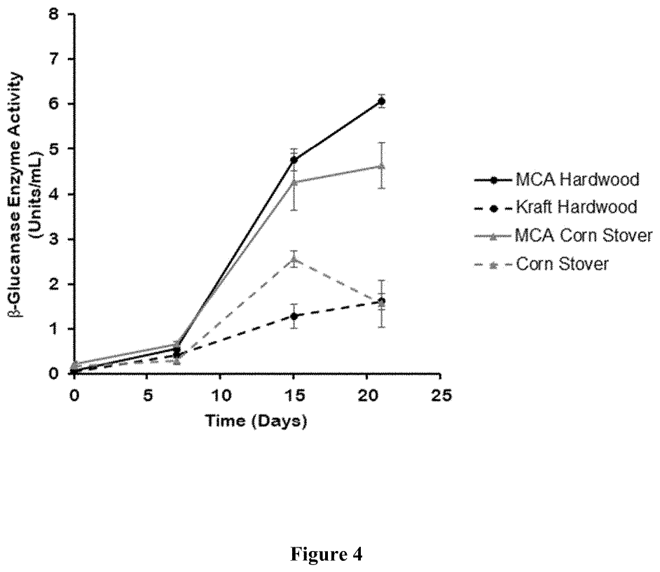

In referring to FIG. 4, one can notice that the MCA cellulose with a lower kappa number have generated higher enzyme activity in the first exposure to a bacterium. This first exposure is meant to hydrolyze the β-1,4-glucosidic bonds of the cellulose to generate smaller cellulose molecules and cellobiose and therefore generate the bulk of the glucose precursor material (in this case, cellobiose). It is understood that the second step of the process according to a preferred embodiment of the present invention is straightforward as it does not deviate from the common approach of cellobiose conversion to glucose. It is established that the first step is more determinant of the extent of biomass conversion to glucose since it will generate as main primary product, the cellobiose, which is subsequently employed in conversion to glucose. It is imperative that the cellulose degradation to cellobiose be maximized at this step, otherwise it will prevent the second step of the process from having a significant impact.

According to a preferred embodiment of the present invention, it is observed that, in the last data point, that the enzymatic activity is higher with the MCA cellulose than the enzymatic activity when such is exposed to a a kraft processed hardwood cellulose (kappa number=15.82) or raw corn stover biomass (kappa number=47.74). The MCA cellulose is obtained according to a process (or approach) to obtain low lignin cellulose (also referred to as a low kappa number cellulose, also referred to as modified Caro's acid delignified cellulose) as prepared according to a preferred process described hereinbelow and has a kappa number=β-2.

### Process to Obtain a Low Kappa Number Delignified Cellulose

According to a preferred embodiment of the present invention, the method of delignification of biomass material which yields a Modified Caro's Acid (MCA) cellulose used in the cellulose to cellobiose (and ultimately, glucose) conversion experiments comprise:
providing a biomass material comprising cellulose fibers and lignin;
exposing said biomass material requiring delignification to a modified Caro's acid composition selected from the group consisting of: composition A; composition B and Composition C;

wherein said composition A comprises:
sulfuric acid in an amount ranging from 20 to 70 wt % of the total weight of the composition;
a compound comprising an amine moiety and a sulfonic acid moiety selected from the group consisting of: taurine; taurine derivatives; and taurine-related compounds; and
a peroxide;

wherein said composition B comprises:
an alkylsulfonic acid; and
a peroxide; wherein the acid is present in an amount ranging from 40 to 80 wt % of the total weight of the composition and where the peroxide is present in an amount ranging from 10 to 40 wt % of the total weight of the composition;

wherein said composition C comprises:
sulfuric acid;
a compound comprising an amine moiety;
a compound comprising a sulfonic acid moiety; and
a peroxide;
   for a period of time sufficient to remove substantially all of the lignin present on said biomass material. The process can be carried out for a varying duration of time depending on the particle size of the biomass being fed into the process. The process can last from 2 to 20 hours depending on that characteristic. Moreover, the temperature of the resulting mixture also has an impact on the duration of the process as the reaction is highly exothermic, precautions are taken to prevent a runaway degradation of the cellulose. This would result in a carbon black resulting product with no value. The process is preferably run at temperatures below 50° C., more preferably at temperatures below 40° C. the process of delignification is preferably performed with a cooling means adapted to control the heat generated by the chemical reaction of delignification and maintain the temperature to avoid an undesirable ‘runaway’ reaction.

Preferably, said sulfuric acid, said compound comprising an amine moiety and a sulfonic acid moiety and said peroxide are present in a molar ratio of no more than 15:1:1.

According to a preferred embodiment of the approach to obtain low lignin cellulose, said sulfuric acid and said compound comprising an amine moiety and a sulfonic acid moiety are present in a molar ratio of no less than 3:1.

Preferably, said compound comprising an amine moiety and a sulfonic acid moiety is selected from the group consisting of: taurine; taurine derivatives; and taurine-related compounds.

According to a preferred embodiment of the approach to obtain low lignin cellulose, said taurine derivative or taurine-related compound is selected from the group consisting of: taurolidine; taurocholic acid; tauroselcholic acid; tauromustine; 5-taurinomethyluridine and 5-taurinomethyl-2-thiouridine; homotaurine (tramiprosate); acamprosate; and taurates; as well as aminoalkylsulfonic acids where the alkyl is selected from the group consisting of C₁-C₅ linear alkyl and C₁-C₅ branched alkyl.

Preferably, said linear alkylaminosulfonic acid is selected form the group consisting of: methyl; ethyl (taurine); propyl; and butyl.

Preferably, branched aminoalkylsulfonic acid is selected from the group consisting of: isopropyl; isobutyl; and isopentyl.

According to a preferred embodiment of the approach to obtain low lignin cellulose, said compound comprising an amine moiety and a sulfonic acid moiety is taurine.

According to a preferred embodiment of the approach to obtain low lignin cellulose, said sulfuric acid and compound comprising an amine moiety and a sulfonic acid moiety are present in a molar ratio of no less than 3:1.

According to a preferred embodiment of the approach to obtain low lignin cellulose, said compound comprising an amine moiety is an alkanolamine is selected from the group consisting of: monoethanolamine; diethanolamine; triethanolamine; and combinations thereof.

According to a preferred embodiment of the approach to obtain low lignin cellulose, said compound comprising a sulfonic acid moiety is selected from the group consisting of: alkylsulfonic acids; arylsulfonic acids; and combinations thereof.

Preferably, said alkylsulfonic acid is selected from the group consisting of: alkylsulfonic acids where the alkyl groups range from C₁-C₆ and are linear or branched; and combinations thereof. More preferably, said alkylsulfonic acid is selected from the group consisting of: methanesulfonic acid; ethanesulfonic acid; propanesulfonic acid; 2-propanesulfonic acid; isobutylsulfonic acid; t-butylsulfonic acid; butanesulfonic acid; iso-pentylsulfonic acid; t-pentylsulfonic acid; pentanesulfonic acid; t-butylhexanesulfonic acid; and combinations thereof.

Preferably, said arylsulfonic acid is selected from the group consisting of: toluenesulfonic acid; benzesulfonic acid; and combinations thereof.

According to a preferred embodiment of the approach to obtain low lignin cellulose, said alkylsulfonic acid; and said peroxide are present in a molar ratio of no less than 1:1.

Preferably, said compound comprising a sulfonic acid moiety is methanesulfonic acid.

According to a preferred embodiment of the approach to obtain low lignin cellulose, said Composition C may further comprise a compound comprising an amine moiety. Preferably, the compound comprising an amine moiety has a molecular weight below 300 g/mol. Preferably also, the compound comprising an amine moiety is a primary amine. More preferably, the compound comprising an amine moiety is an alkanolamine. Preferably, the compound comprising an amine moiety is a tertiary amine. According to a preferred embodiment of the approach to obtain low lignin cellulose, the alkanolamine is selected from the group consisting of: monoethanolamine; diethanolamine; triethanolamine; and combinations thereof. Preferably, the alkanolamine is triethanolamine.

According to a preferred embodiment of the approach to obtain low lignin cellulose, said in Composition C, said sulfuric acid and said a compound comprising an amine moiety and said compound comprising a sulfonic acid moiety are present in a molar ratio of no less than 1:1:1.

Preferably, in Composition C, said sulfuric acid, said compound comprising an amine moiety and said compound comprising a sulfonic acid moiety are present in a molar ratio ranging from 28:1:1 to 2:1:1.

Preferably, in Composition C, said compound comprising an amine moiety is triethanolamine and said compound comprising a sulfonic acid moiety is methanesulfonic acid.

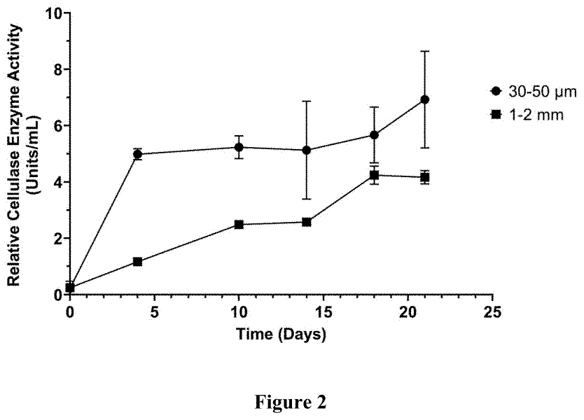

In referring to FIG. 2, which illustrates a comparative graphical representation of the enzymatic activity of an adapted strain of Cellulomonas uda (ATCC491) between fine grade MCA cellulose (30-50 μm) and coarse grade MCA cellulose (1-2 mm) according to a preferred embodiment of the present invention, both of which are obtained from the process described hereinabove. It is worth noting that the particle size of the cellulose feedstock has a non-negligible impact on the enzymatic activity of the bacterium used in this series of experiments.

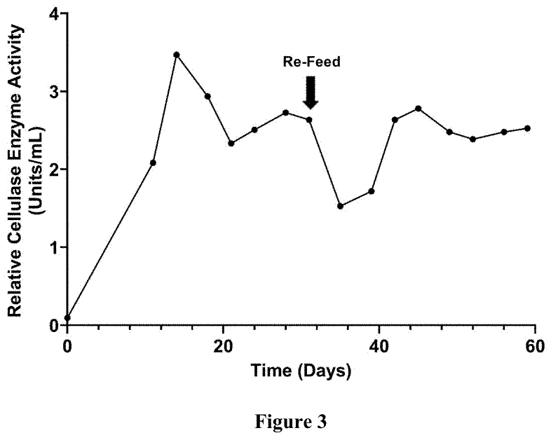

In referring to FIG. 3, which illustrates a graphical representation of the enzymatic activity of the original strain of Cellulomonas Uda (ATCC491) in a continuous feed model according to a preferred embodiment of the present invention, it is noted that after re-feeding of the culture the lag phase is minimal and it takes 5-7 days for the organism to return to the enzymatic activity seen in the first culture. This demonstrates that a continuous feed culture model will allow for enzyme activity to remain high and reduce lag periods that are seen with batch or fed-batch culture systems.

In referring to FIG. 4, which illustrates a comparative graphical representation of the enzymatic activity of an adapted strain of Cellulomonas uda (ATCC491), after a few generations of exposure to different grades of cellulose, it is noted that the bacterium exhibits very high affinity for the MCA cellulose obtained from a process as described hereinabove. This is surprising knowing the fact that, as mentioned above, the enzymatic hydrolysis of a cellulase bacterium is typically hindered by the level of crystallinity of the cellulose it is exposed to. The conclusion upon reviewing the graph is that the kappa number of a source of cellulose has a direct and non-negligible impact on the ability of the adapted strain of Cellulomonas uda (ATCC491) to metabolize cellulose into cellobiose.

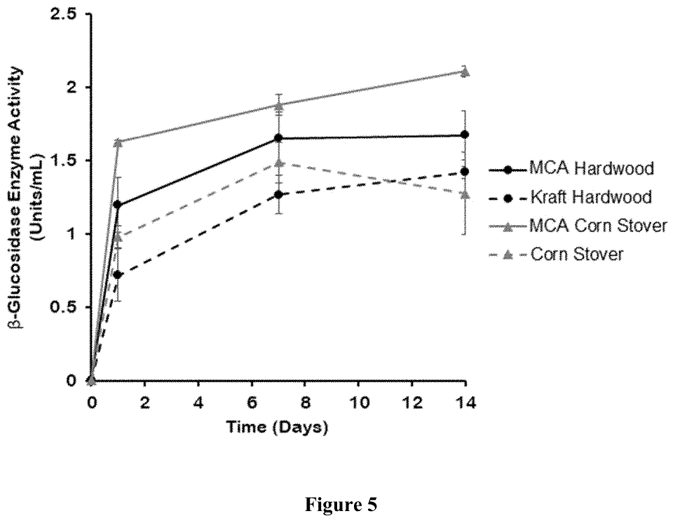

FIG. 5 shows the enzyme activity of beta-glucosidase (from Aspergillus brasiliensis) is much greater in the modified Caro's acid delignified cellulose samples compared to those made by the kraft process, increasing by approximately 20% in hardwood and 65% in corn stover. This enzymatic reaction is converting the cellobiose to glucose. This second reaction occurs in a semi-anoxic co-culture of Aspergillus brasiliensis and Saccharomyces cerevisiae (yeast), so that as A. brasiliensis converts the cellobiose to glucose, the yeast can then ferment the glucose to ethanol.

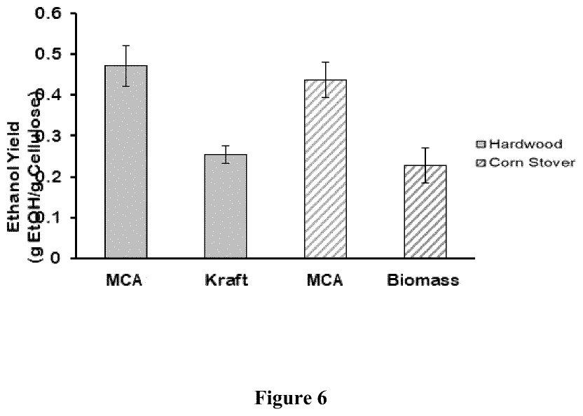

FIG. 6 shows the ethanol yields obtained per gram of cellulose. Ethanol yields from a process using a modified Caro's acid and kraft process are also listed in the table below. Using the modified Caro's acid delignification process, ethanol yields increased by 85% in hardwood (compared to the kraft) and 92% in corn stover (compared to raw biomass).

TABLE 1
Ethanol yield from cellulose obtained
from various source material using
either the Kraft delignification process
or using a modified Caro’s acid

| | g (EtOH)/ |
|---|---|
| Sample | g (cellulose) |
| Hardwood (pref. emb.) | 0.47 |
| Hardwood (Kraft process) | 0.25 |
| Corn stover (pref. emb.) | 0.44 |
| Corn stover (Kraft process) | 0.23 |

According to a preferred embodiment of the process of the present invention, the step of exposing the adapted strain of Cellulomonas uda, (ATCC491) to a source of cellulose generates over 40% more enzymatic activity than the same bacterium on a kraft pulp with a kappa number of 27. Moreover, in the same circumstance, the β-1,4-glucanase enzyme activity (in unit per ml) was approximately 38% more active when exposed to a cellulose having aspect ratio of 7.5, a particle size ranging from 30 to 50 μm and a kappa number ranging from β-2 than when it was exposed to a kraft pulp where the cellulose has a kappa number of 27.

According to a preferred embodiment of the process of the present invention, this process will enable a higher conversion of cellulose to glucose by overcoming the first step in the chain of reactions which is the conversion of cellulose to cellobiose.

Given this information, it is believed that idle ethanol plants located around the world could re-start operations of cellulose conversion to glucose (and subsequently, ethanol) if a biomass feedstock according to the following specifications was employed rather than using corn, sugar cane or conventional kraft pulp. Moreover, the implementation of a process according to a preferred embodiment of the present invention would essentially “dovetail” with the delignification process of a lignocellulosic biomass by using a modified Caro's acid, and the production of ethanol with the cellulose obtained from the delignification process. As mentioned previously, the person skilled in the art will recognize that by employing a cellulose obtained from a process using a modified Caro's acid, one will circumvent the need of any further or subsequent bleaching step following the delignification. It is to be understood that the bleaching refers to a separate and distinct step of pulp processing. Consequently, the pulp used obtained using a modified Caro's acid driven delignification process, is intended on being a pulp which has not undergone a separate bleaching step post-delignification. As is also understood by the person skilled in the art, such a treatment step (bleaching) is understood to not be economically viable when the ultimate goal of the cellulose is to be further converted in order to generate ethanol.

While the foregoing invention has been described in some detail for purposes of clarity and understanding, it will be appreciated by those skilled in the relevant arts, once they have been made familiar with this disclosure that various changes in form and detail can be made without departing from the true scope of the invention in the appended claims.

1. A process to hydrolyze cellulose into cellobiose, said process comprising the following steps:
providing a reaction vessel;
providing a source of cellulose into said reaction vessel; wherein said source of cellulose has a kappa number of less than 10;
providing a Cellulomonas uda (ATCC 491) inoculum into said reaction vessel;
exposing said Cellulomonas uda (ATCC 491) to said source of cellulose in an aqueous medium of pH of about 8 at a temperature ranging from 30° C. to 35° C. for a period of time ranging from 14 to 42 days; and
optionally, recovering the supernatant comprising cellobiose to be converted to glucose and or ethanol using a bacterium, fungi, yeast or combination.

2. Process according to claim 1, wherein said source of cellulose has a particle size ranging between 30-50 μm.

3. The process according to claim 1 further comprising a step of exposing the cell supernatant to a bacterium or fungi or yeast, or combination to convert cellobiose to glucose or ethanol.

4. The process according to claim 1, wherein the source of cellulose is a lignocellulosic biomass delignified by exposure to a modified Caro's acid composition selected from the group consisting of: composition A; composition B and Composition C;
wherein said composition A comprises:
sulfuric acid in an amount ranging from 20 to 70 wt % of the total weight of the composition;
a compound comprising an amine moiety and a sulfonic acid moiety selected from the group consisting of: taurine; taurine derivatives; and taurine-related compounds; and
a peroxide;
wherein said composition B comprises:
an alkylsulfonic acid; and
a peroxide; wherein the acid is present in an amount ranging from 40 to 80 wt % of the total weight of the composition and where the peroxide is present in an amount ranging from 10 to 40 wt % of the total weight of the composition;
wherein said composition C comprises:
sulfuric acid;
a compound comprising an amine moiety;
a compound comprising a sulfonic acid moiety; and
a peroxide;
for a period of time sufficient to remove substantially all of the lignin present on said biomass material.

5. The process according to claim 4, wherein said sulfuric acid, said compound comprising an amine moiety and a sulfonic acid moiety and said peroxide are present in a molar ratio of no more than 15:1:1.

6. The process according to claim 4, wherein said sulfuric acid and said compound comprising an amine moiety and a sulfonic acid moiety are present in a molar ratio of no less than 3:1.

7. The process according to claim 4, wherein compound comprising an amine moiety and a sulfonic acid moiety is selected from the group consisting of: taurine; taurine derivatives; and taurine-related compounds.

8. The process according to claim 4, wherein taurine derivative or taurine-related compound is selected from the group consisting of: taurolidine; taurocholic acid; tauroselcholic acid; tauromustine; 5-taurinomethyluridine and 5-taurinomethyl-2-thiouridine; homotaurine (tramiprosate); acamprosate; and taurates; as well as aminoalkylsulfonic acids where the alkyl is selected from the group consisting of C₁-C₅ linear alkyl and C₁-C₅ branched alkyl.

9. The process according to claim 8, wherein linear alkylaminosulfonic acid is selected form the group consisting of: methyl; ethyl (taurine); propyl; and butyl.

10. The process according to claim 4, wherein said compound comprising an amine moiety and a sulfonic acid moiety is taurine.

11. The process according to claim 4, wherein said alkylsulfonic acid is selected from the group consisting of: methanesulfonic acid; ethanesulfonic acid; propanesulfonic acid; 2-propanesulfonic acid; isobutylsulfonic acid; t-butylsulfonic acid; butane sulfonic acid; iso-pentylsulfonic acid; t-pentylsulfonic acid; pentanesulfonic acid; t-butylhexanesulfonic acid; and combinations thereof.

12. The process according to claim 4, where, in Composition C, said compound comprising an amine moiety is an alkanolamine selected from the group consisting of: monoethanolamine; diethanolamine; triethanolamine; and combinations thereof.

13. The process according to claim 4 where, in Composition C, said compound comprising an amine moiety is triethanolamine and said compound comprising a sulfonic acid moiety is methanesulfonic acid.

14. The process according to claim 4, wherein the delignification lasts from 2 to 20 hours.

15. The process according to claim 4, wherein the delignification is carried out at temperatures below 50° C.

16. The process according to claim 4, wherein said cellulose is characterized by an absence of prior exposure to bleaching chemicals selected from the group consisting of: sodium hydrosulphite (Na₂S₂O₄); hydrogen peroxide; pentasodium salt diethylenetriaminepentaacetic acid; amine borane (CH₃)₃CNH₂-BH₃; borane ammonia complex BH₃—NH3; sodium percarbonate; formamidine sulphinic acid; sodium perborate; and chlorine dioxide.

17. A process to convert cellulose to glucose or ethanol, said process comprising the steps of:
providing a reaction vessel;
providing a source of cellulose into said reaction vessel; wherein said source of cellulose having a particle size ranging between 30-50 μm and a kappa number of less than 10;
providing a Cellulomonas uda (ATCC 491) inoculum into said reaction vessel;
exposing said Cellulomonas uda (ATCC 491) to said source of cellulose in an aqueous medium of pH of about 8 at a temperature ranging from 30° C. to 35° C. for a period of time ranging from 14 to 42 days, allowing sufficient exposure time to convert at least 90% of the cellulose into cellobiose and/or glucose; and
optionally, recovering the supernatant comprising cellobiose and glucose.

18. The process according to claim 17, where the temperature inside the reaction vessel during said exposure time does not exceed 60° C.

19. The process according to claim 17, wherein said cellulose has an aspect ratio of 7.5.

20. A process to obtain a bacterium having a high cellulase activity for a low kappa number cellulose, wherein said a low kappa number cellulose has the following characteristics: particle size ranging from 30-50 microns and a kappa number of less than 10, more preferably less than 5 and even more preferably, less than 2, wherein said process comprising the steps of:
providing a reaction vessel;
providing a Cellulomonas uda (ATCC491) inoculum which has a cellulase enzyme activity when exposed to said low kappa number cellulose of less than 5 units/ml after said bacterium has been exposed to said low kappa number cellulose for 21 days;
exposing said Cellulomonas uda (ATCC491) bacterium to said low kappa number cellulose in an aqueous medium of pH of about 8 at a temperature ranging from 30° C. to 35° C. for a period of time ranging from 14 to 42 days;
recovering the bacterium and repeating the step of exposing said bacterium to said low kappa number cellulose for a number of cycles of exposure and recovery until the cellulase activity of the enzyme reaches at least 8 units/ml when said bacterium is exposed to said low kappa number cellulose for at least 18 days, wherein said bacterium has been optimized for cellulose hydrolysis; and
optionally, recovering the optimized bacterium.
optionally, removing the products and refeeding the culture for a continuous batch culture.
